(19) United States
(12) Patent Application Publication
(10) Pub. No.: US 2022/0008156 A1
(43) Pub. Date: Jan. 13, 2022

(54) SURGICAL OBSERVATION APPARATUS, SURGICAL OBSERVATION METHOD, SURGICAL LIGHT SOURCE DEVICE, AND SURGICAL LIGHT IRRADIATION METHOD

(71) Applicant: Sony Corporation, Tokyo (JP)

(72) Inventor: Kei TOMATSU, Tokyo (JP)

(73) Assignee: Sony Corporation, Tokyo (JP)

(21) Appl. No.: 17/052,215

(22) PCT Filed: Jun. 3, 2019
(86) PCT No.: PCT/JP2019/022009

§ 371 Date: Nov. 2, 2020

(30) Foreign Application Priority Data
Jun. 15, 2018 (JP) 2018-114592

(51) Int. Cl. A61B 90/30; A61B 90/00

(52) U.S. Cl. CPC A61B 90/30; A61B 1/0638; A61B 90/361

(57) ABSTRACT

The present disclosure is to provide a surgical observation apparatus (2000) that includes: a light source unit (1000) including a first light source (198) that emits observation light for observing an operative field, a second light source (100, 110, 120, 130, 140, 150) that emits special light in a wavelength band different from the first light source; and an optical system (190) capable of changing the emission angle of the special light with respect to the operative field, the light source unit emitting the observation light and the special light onto the operative field from the same emission port; and an imaging unit (2010) that captures an image of the operative field illuminated by the light source unit.

### TECHNICAL FIELD

The present disclosure relates to a surgical observation apparatus, a surgical observation method, a surgical light source device, and a method in a surgical light irradiation method.

### BACKGROUND ART

Patent Document 1 listed below as a conventional art discloses an imaging apparatus, an imaging system, a surgical navigation system, and an imaging method capable of capturing an image of an object including a fluorescent substance with high precision in a short exposure time, for example. Patent Document 1 discloses a method for conducting special light observation by zooming the imaging region and changing the size of the illumination region in conjunction with the change in the imaging region.

### CITATION LIST

### Patent Document

Patent Document 1: Japanese Patent Application Laid-Open No. 2012-23492

### SUMMARY OF THE INVENTION

### Problems to be Solved by the Invention

For example, a method for emitting special light for exciting a fluorescent agent by introducing a biomarker such as a fluorescent agent into an observation target such as an organ is used these days. However, fluorescence generated by excitation light is weak in some cases, and it might become necessary to zoom the imaging region to clearly recognize the fluorescence in the close observation region. For example, by the method disclosed in Patent Document 1 mentioned above, to closely observe the close observation region and the closely observed region by zooming, it is necessary to enlarge the screen display area by zooming or the like. Here, by the method disclosed in Patent Document 1 mentioned above, the region to be observed with special light is zoomed, and therefore, the surrounding regions cannot be observed during the zooming.

Further, by the method disclosed in Patent Document 1 mentioned above, in a case where zooming is performed after the region the observer wishes to observe closely is determined, the special light is also emitted onto regions not displayed on the screen. Because of this, in a case where the observation target is an internal organ or the like of the human body, for example, there is a possibility that regions not being observed with the special light will be damaged.

Therefore, there is a demand for optimization of the irradiation region of the special light.

### Solutions to Problems

The present disclosure is to provide a surgical observation apparatus that includes: a light source unit including a first light source that emits observation light for observing an operative field, a second light source that emits special light in a wavelength band different from the first light source, and an optical system capable of changing the emission angle of the special light with respect to the operative field, the light source unit emitting the observation light and the special light onto the operative field from the same emission port; and an imaging unit that captures an image of the operative field illuminated by the light source unit.

The present disclosure is also to provide a surgical observation method that includes: emitting observation light for observing an operative field; emitting special light in a wavelength band different from the observation light; emitting the observation light and the special light onto the operative field from the same emission port; changing the emission angle of the special light with respect to the operative field; and capturing an image of the operative field illuminated by the observation light and the special light.

The present disclosure is further to provide a surgical light source device that includes: a first light source that emits observation light for observing an operative field; a second light source that emits special light in a wavelength band different from the first light source; and an optical system capable of changing the emission angle of the special light with respect to the operative field. The observation light and the special light are emitted onto the operative field from the same emission port.

The present disclosure is also to provides a surgical light irradiation method that includes: emitting observation light for observing an operative field; emitting special light in a wavelength band different from the observation light; emitting the observation light and the special light onto the operative field from the same emission port; and changing the emission angle of the special light with respect to the operative field.

### Effects of the Invention

As described above, according to the present disclosure, it is possible to optimize the irradiation region of special light. Note that the effect described above is not necessarily restrictive, and it is possible to achieve any one of the effects described in this specification together with the effect described above or instead of the effect described above, or it is possible to achieve other effects obvious from this specification.

### BRIEF DESCRIPTION OF DRAWINGS

FIG. 1 is a schematic diagram showing the configuration of a light source device according to an embodiment of the present disclosure.

FIG. 2 is a schematic diagram showing another example of a light source device.

FIG. 3 is a schematic diagram showing a screen display area that is captured and displayed by the camera of an endoscope.

FIG. 4 is a schematic diagram for explaining zooming with special light.

FIG. 5 is a schematic diagram showing the relationship between the screen display area, and the irradiation region of visible light and the irradiation region of the special light.

FIG. 6 is a schematic diagram showing the relationship between the screen display area, and the irradiation region of the visible light and the irradiation region of the special light.

FIG. 7 is a schematic diagram showing the exterior of the light source device.

FIG. 8 is a schematic diagram showing the configuration of a surgical observation apparatus to which the light source device is applied.

FIG. 9 is an explanatory diagram for explaining a system for medical use to which the light source device according to an embodiment of the present disclosure is applied.

FIG. 10 is a schematic view showing the exterior of the robot arm device shown in FIG. 9.

FIG. 11 is a diagram schematically showing an example configuration of an endoscopic surgery system.

FIG. 12 is a block diagram showing an example of the functional configurations of a camera head and a CCU shown in FIG. 11.

### MODE FOR CARRYING OUT THE INVENTION

The following is a detailed description of preferred embodiments of the present disclosure, with reference to the accompanying drawings. Note that, in this specification and the drawings, components having substantially the same functional configurations are denoted by the same reference numerals, and explanation of them will not be repeated.

Note that explanation will be made in the following order.

1. Configuration of a light source device

2. Special light zooming

3. Exterior of the light source device

4. Example configuration of a surgical observation apparatus

5. Example configuration of a system for medical use

6. Variation of control

### 1. Configuration of a Light Source Device

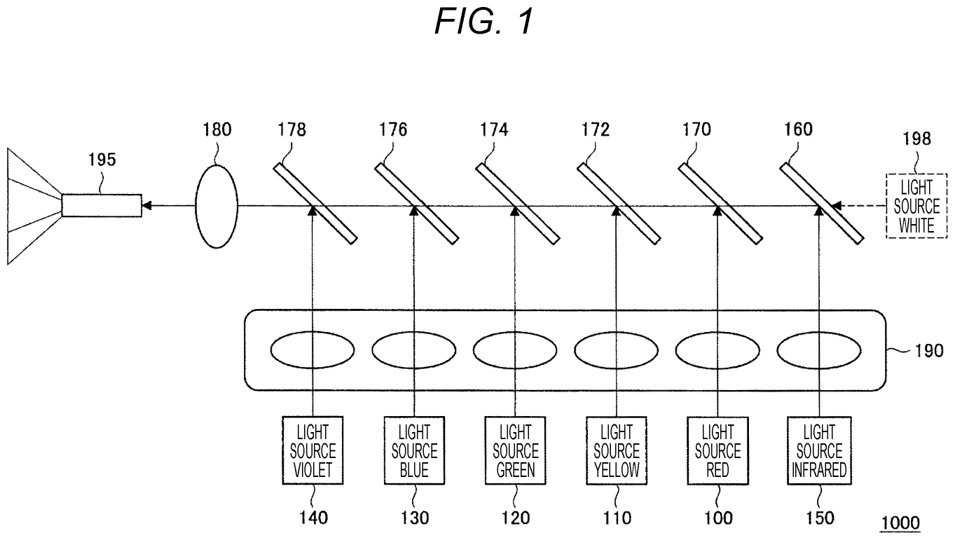

FIG. 1 is a schematic diagram showing the configuration of a light source device 1000 according to an embodiment of the present disclosure. The light source device 1000 is applied to a system for medical use such as an endoscope system or a microscope system, and emits visible light (white light) onto an observation target imaged by an imaging apparatus, and excitation light (hereinafter referred to as special light) for exciting a fluorescent agent (a contrast agent), from the same emission port. Note that, for ease of explanation, a case where the light source device 1000 is applied mainly to an endoscope system will be described as an example in the description below.

As shown in FIG. 1, the light source device 1000 includes a red light source 100, a yellow light source 110, a green light source 120, a blue light source 130, a violet light source 140, an infrared light source 150, a mirror 160, dichroic mirrors 170, 172, 174, 176, and 178, a condenser lens 180, a zoom optical system 190, and a light guide 195.

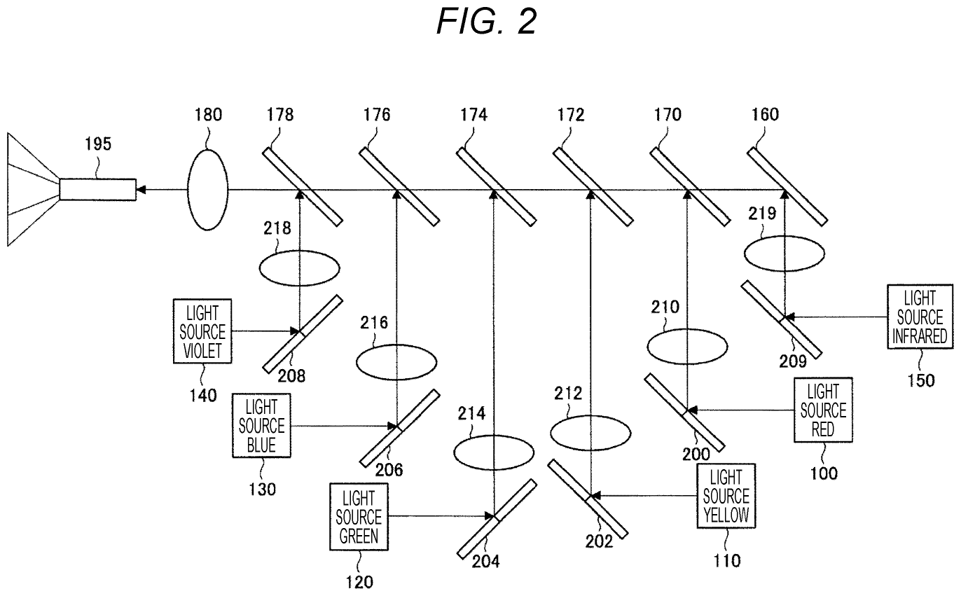

Further, FIG. 2 is a schematic diagram showing another example of the light source device 1000. The configuration including the red light source 100, the yellow light source 110, the green light source 120, the blue light source 130, the violet light source 140, the infrared light source 150, the mirror 160, the dichroic mirrors 170, 172, 174, 176, and 178, and the condenser lens 180 is similar to that shown in FIG. 1. In the configuration shown in FIG. 2, instead of the zoom optical system 190 shown in FIG. 1, zoom split mirrors 200, 202, 204, 206, 208, and 209, and zoom condenser lenses 210, 212, 214, 216, 218, and 219 are provided.

As shown in FIGS. 1 and 2, infrared light emitted from the infrared light source 150 is reflected by the mirror 160 at an angle of 90 degrees, is transmitted through the dichroic mirrors 170, 172, 174, 176, and 178, and is condensed by the condenser lens 180. Red light from the red light source 100 is emitted toward the dichroic mirror 170, yellow light from the yellow light source 110 is emitted toward the dichroic mirror 172, green light from the green light source 120 is emitted toward the dichroic mirror 174, blue light from the blue light source 130 is emitted toward the dichroic mirror 176, and violet light from the violet light source 140 is emitted toward the dichroic mirror 178. Note that, in the configuration shown in FIG. 2, light emitted from the respective light sources is reflected by the respective zoom split mirrors, is condensed by the respective zoom condenser lenses, and is then emitted to the respective dichroic mirrors.

The dichroic mirror 170 has such optical characteristics as to reflect only the red wavelength. The dichroic mirror 172 has such optical characteristics as to reflect only the yellow wavelength. The dichroic mirror 174 has such optical characteristics as to reflect only the green wavelength. The dichroic mirror 176 has such optical characteristics as to reflect only the blue wavelength. The dichroic mirror 178 has such optical characteristics as to reflect only the violet wavelength.

The infrared wavelength from the infrared light source 150 is combined with the red wavelength from the red light source 100 at the dichroic mirror 170, is combined with the blue wavelength from the yellow light source 110 at the dichroic mirror 172, is combined with the green wavelength from the green light source 120 at the dichroic mirror 174, is combined with the blue wavelength from the blue light source 130 at the dichroic mirror 176, and is combined with the violet wavelength from the violet light source 140 at the dichroic mirror 178. The combined light is condensed at the condenser lens 180. The light condensed at the condenser lens 180 passes through the light guide 195 and is emitted onto the observation target. Note that an observation optical system for refracting light emitted from the light guide 195 may be further provided. As the infrared wavelength, the red wavelength, the yellow wavelength, the green wavelength, the blue wavelength, and the violet wavelength are combined as described above, it is possible to emit white visible laser light from the condenser lens 180.

In FIG. 1, each ray of light emitted from the red light source 100, the yellow light source 110, the green light source 120, the blue light source 130, the violet light source 140, and the infrared light source 150 is enlarged or reduced by the zoom optical system 190. Note that the configuration of the zoom optical system 190 may be a general-purpose one, and, for example, the configuration disclosed in Japanese Patent Application Laid-Open No. 2013-37105 or the like can be used as appropriate.

Meanwhile, in FIG. 2, the respective rays of light emitted from the red light source 100, the yellow light source 110, the green light source 120, the blue light source 130, the violet light source 140, and the infrared light source 150 are enlarged or reduced by the zoom split mirrors 200, 202, 204, 206, 208, and 209. The respective rays of light enlarged or reduced by the zoom split mirrors 200, 202, 204, 206, 208, and 209 are condensed by the zoom condenser lenses 210, 212, 214, 216, 218, and 219, and enter the dichroic mirrors 170, 172, 174, 176, and 178 and the mirror 160. Note that the configuration disclosed in WO 2018/029962 A, for example, can be used as the configuration of the zoom split mirrors 200, 202, 204, 206, 208, and 209.

Note that the zoom optical system 190 or the zoom split mirrors 200, 202, 204, 206, 208, and 209 may not be provided for all the light sources, but may be provided only for the light sources in the wavelength band to be used for exciting the fluorescent agent. For example, in a case where the red light source 100, the yellow light source 110, the green light source 120, and the blue light source 130 are used only for generating visible light, the zoom optical system or the zoom split mirrors corresponding to these light sources may not be provided.

### 2. Special Light Zooming

In this embodiment, the irradiation region of light of a predetermined color to be used as special light can be changed by the zoom optical system 190 or the zoom split mirrors 200, 202, 204, 206, 208, and 209 shown in FIGS. 1 and 2. For example, in a case where red light is used as the special light, only red light can be emitted only onto the central portion of the imaging region. In this case, visible light obtained by combining light rays of other colors illuminates the entire imaging region.

Specifically, the irradiation region of the light source of the special light having a wavelength compatible with the fluorescent agent introduced into the observation target is changed. By enlarging or reducing the special light that excites the fluorescent agent, it is possible to emit the special light only onto the necessary region. Thus, the visibility of the observation target can be increased, while damage to the observation target can be reduced. Note that any light from the red light source 100, the yellow light source 110, the green light source 120, the blue light source 130, the violet light source 140, and the infrared light source 150 can be used as the special light. However, light rays from the red light source 100, the yellow light source 110, the green light source 120, and the blue light source 130 also form visible light when combined, and therefore, there is a possibility that the tint of the surrounding region will change in a case where zooming is performed. In this case, to reduce the change in the tint of the surrounding region, a dichroic mirror may be employed in place of the mirror 160 shown in FIG. 1, and a light source 198 that emits white light toward the dichroic mirror may be separately provided. In this case, the special light is combined with visible light emitted from the light source 198. Since the visible light is emitted from the light source 198, it is possible to reduce the change in the tint of the surrounding region in a case where special light zooming is performed.

Particularly, according to this embodiment, it is possible to emit only the special light only onto an affected site such as an organ or a tumor, while emitting visible light onto the entire imaging region. Thus, it is possible to visually recognize in detail the affected site onto which fluorescence is emitted, while visually observing the entire imaging region.

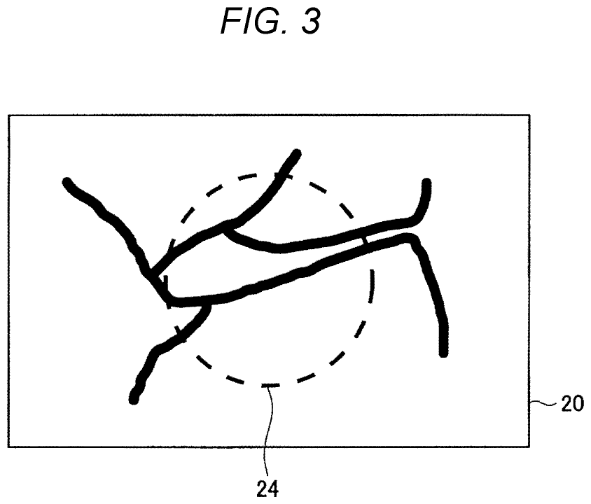

FIG. 3 is a schematic diagram showing a screen display area 20 that is captured and displayed by the camera of an endoscope. The screen display area 20 shows the imaging region captured by the camera. As shown in FIG. 3, the irradiation region 24 after zooming with the special light is displayed with a dashed line in the center of the screen display area 20. As the irradiation region 24 after zooming with the special light is displayed in the screen display area 20, the observer can recognize the post-zooming irradiation region 24 prior to the zooming. Further, as the zooming with the special light is performed, it is possible to enhance the visibility of excitation light within the irradiation region 24, and reduce damage to the observation target outside the irradiation region 24.

The system according to this embodiment can also be set to normal mode in which no special light is to be emitted. The normal mode is selected for normal observation in which no fluorescent agent is introduced into the observation target. In a case where a fluorescent agent is introduced into the observation target, and fluorescence is excited by irradiation of the special light to observe the observation target, on the other hand, special light emission mode for emitting the special light is selected. The observer can switch between the normal mode and the special light emission mode by operating a mode selection switch of the light source device 1000. Note that, in the normal mode, the light sources that are not particularly necessary for generating visible light, such as the violet light source 140, may be turned off, for example. In the special light emission mode, the light sources that are not particularly necessary for exciting the fluorescent agent may also be turned off.

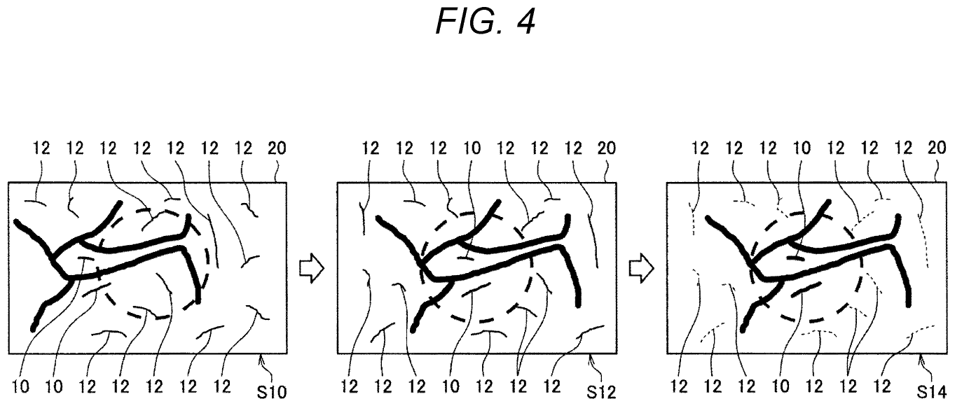

FIG. 4 is a schematic diagram for explaining how zooming is performed with the special light, and shows a situation where the observation target is displayed in the screen display area 20. Note that, as a premise, the special light emission mode is set. Visible light is emitted onto a region including the screen display area 20. As a result, the entire screen display area 20 can be illuminated.

Step S10 in FIG. 4 shows a situation where the observation target displayed in the screen display area includes a region 10 that the observer wishes to closely observe, and also shows a situation where the fluorescence of the region 10 to be closely observed is emitting light from the fluorescent substance introduced into the observation target. This state corresponds to a state in which the observer has set the field of the endoscope at a position where the fluorescent agent is expected to react. The irradiation region of the special light is the same as that of visible light, and imaging is performed in a wide zoom state. As the wide zoom state is set, a location where the excitation light is strong can be searched for in a wider region. Also, as the wide zoom state is set, the intensity of the special light becomes lower, and thus, damage to the observation target can be reduced.

Here, the region 10 to be closely observed is an affected site such as a specific organ or tumor, for example. In the screen display area 20, in addition to the region 10 to be closely observed, there exists an excitation light portion 12 from which a fluorescent substance emits light.

The observer who has found the region 10 to be closely observed in the screen display area 20 in step S10 operates the endoscope, to move the region 10 to be closely observed to the center of the screen display area 20, and fix the field of view. Step S12 shows a state in which the region 10 to be closely observed has moved to the center of the screen display area 20.

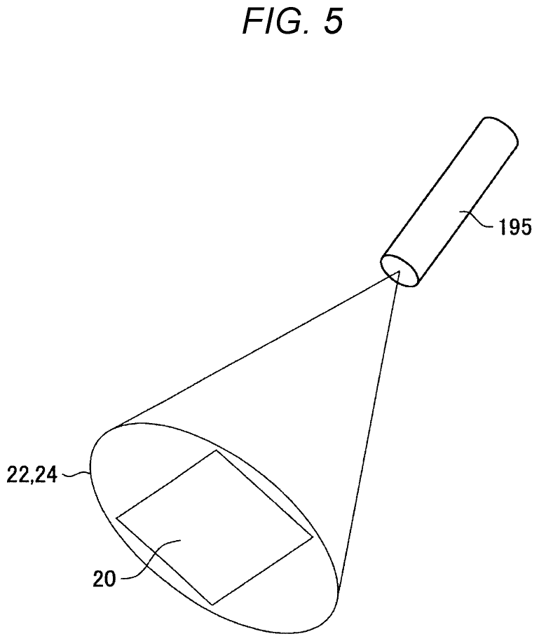

In steps S10 and S12, the irradiation regions of the visible light and the special light are the same. That is, the special light of the color having the wavelength that excites fluorescence is not expanded by the zoom optical system 190 or the zoom split mirrors 200, 202, 204, 206, and 208. FIG. 5 is a schematic diagram showing the relationship between the screen display area 20, and the irradiation region 22 of the visible light and the irradiation region 24 of the special light in steps S10 and S12. As shown in FIG. 5, the irradiation region 22 of the visible light and the irradiation region 24 of the special light are the same, and are larger than the screen display area 20. As described above, in steps S10 and S12, the region 10 to be closely observed is being searched for. Therefore, the irradiation regions of the visible light and the special light are the same, and the special light is emitted onto the entire screen display area 20, so that the search for the region 10 to be closely observed is made easier for the user.

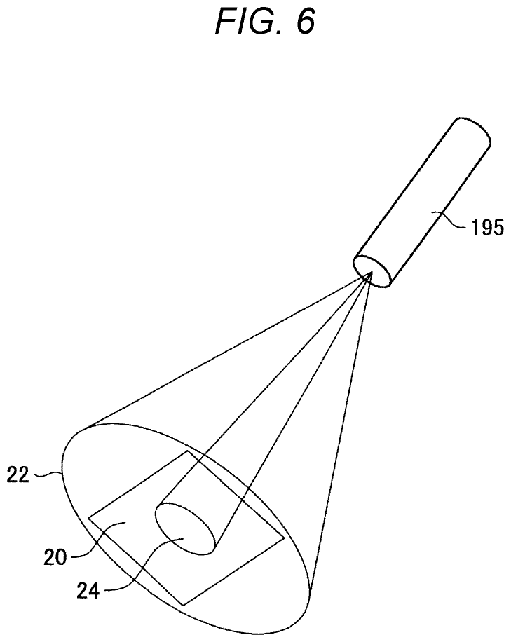

Next, in step S14 in FIG. 4, zoom emission of the special light is performed, so that the irradiation region 24 of the special light becomes smaller than the irradiation region 22 of the visible light. Zooming with the special light is performed by the observer operating a zoom button (an operation unit 310 described later) of the light source device 1000. As a result, only the special light is concentrated in the center of the screen display area 20. FIG. 6 is a schematic diagram showing the relationship between the screen display area 20, and the irradiation region 22 of the visible light and the irradiation region 24 of the special light in step S14. As shown in FIG. 6, the irradiation region 22 of the visible light is the same as that in FIG. 5, but the irradiation region 24 of the special light is smaller than that in FIG. 5, and is concentrated in the center of the screen display area 20.

In FIG. 4, the irradiation region 24 after zooming with the special light is displayed with dashed lines, as in FIG. 3. As the irradiation region 24 after zooming with the special light is displayed in the screen display area 20, the observer can predict the post-zooming irradiation region 24 prior to the zooming. In a case where the irradiation region 24 can be made smaller stepwise to a plurality of sizes, a plurality of irradiation regions 24 may be displayed with dashed lines. In a case where the irradiation region 24 can be continuously made smaller, on the other hand, the minimum irradiation region 24 may be displayed with a dashed line. As the irradiation region 24 of the special light is made smaller, the excitation light becomes stronger at the central portion of the screen display area 20, and becomes weaker at the peripheral portion. Accordingly, the excitation light in the region 10 to be closely observed becomes stronger, so that the region 10 to be closely observed can be observed in detail. As for the excitation light portion 12 outside the region 10 to be closely observed, on the other hand, the excitation light becomes weaker, and thus, damage to the excitation light portion 12 due to the special light can be reduced.

The series of operations shown in FIG. 4 enables the observer to readily find a place where the excitation light is strong by illuminating a wide region with the special light when starting the special light emission. Further, after the region 10 to be closely observed is successfully found, a clear excitation light image can be obtained through zoom emission of only the special light. Note that, although FIG. 4 shows a case where zooming is performed only with the special light, the visible light may also be used in zooming together with the special light.

In a case where the special light is set in a wide state, the intensity of the special light drops relatively. Therefore, to observe the excitation light, setting for increasing sensitivity is performed, such as opening the camera diaphragm or maximizing the ISO sensitivity. In this case, image noise also increases. In a case where zooming is performed with the special light, and the irradiation region 24 is set at the center of the screen as in step S14 in FIG. 4, the intensity of the special light becomes higher. Accordingly, there is no need to increase sensitivity on the camera side, and it is possible to obtain a clear image by reducing generation of noise. As the irradiation region of the special light is changed in this manner, the intensity of the special light is changed. Thus, an optimum image can be obtained. Meanwhile, it is also possible to adjust image quality by changing the gain in sensitivity on the camera side. Thus, according to this embodiment, it is possible to perform observation with an optimum image quality, using both the change of the irradiation region of the special light and the adjustment of the gain in sensitivity on the camera side.

### 3. Exterior of the Light Source Device

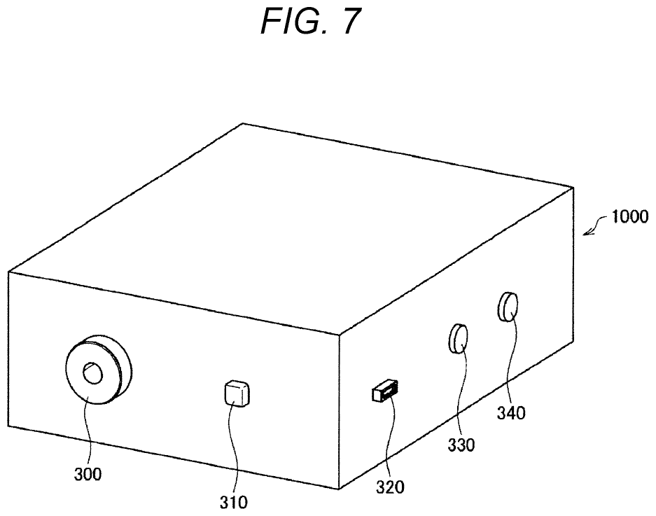

FIG. 7 is a schematic diagram showing the exterior of the light source device 1000. As shown in FIG. 7, the light source device 1000 includes an irradiation port 300 that emits visible light and special light. The light guide 195 extends outward from the irradiation port 300 to the vicinity of the observation target. The light source device 1000 also includes an operation unit 310 for changing the irradiation region of the special light. As the observer operates the operation unit 310, control information for controlling the zoom optical system 190 or the zoom split mirrors 200, 202, 204, 206, 208, and 209 is input via the operation unit 310, and the irradiation region 24 of the special light can be enlarged or reduced.

The light source device 1000 also includes a communication connector 320. Control information for controlling the zoom optical system 190 or the zoom split mirrors 200, 202, 204, 206, 208, and 209 is input to the light source device 1000 via a communication cable connected to the communication connector 320. As the control information is input from the communication connector 320, it is possible to enlarge or reduce the irradiation region 24 of the special light.

The light source device 1000 also includes a mode selection switch 330 and a special light selection switch 340. As described above, the observer can switch between the normal mode and the special light emission mode by operating the mode selection switch of the light source device 1000. Meanwhile, the special light selection switch 340 is a switch for selecting the light source to be used as the special light. As described above, in this embodiment, the irradiation region of the light source of the special light having a wavelength compatible with the fluorescent agent introduced into the observation target is changed. The observer can select the light source compatible with the fluorescent agent by operating the special light selection switch 340. Further, the wavelength of the special light is compatible with the type of the fluorescent agent. Accordingly, the observer may operate the special light selection switch 340 so that the type of fluorescent agent is selected. In this case, the light source device 1000 selects the light source compatible with the fluorescent agent, in accordance with the selected type of fluorescent agent.

### 4. Example Configuration of a Surgical Observation Apparatus

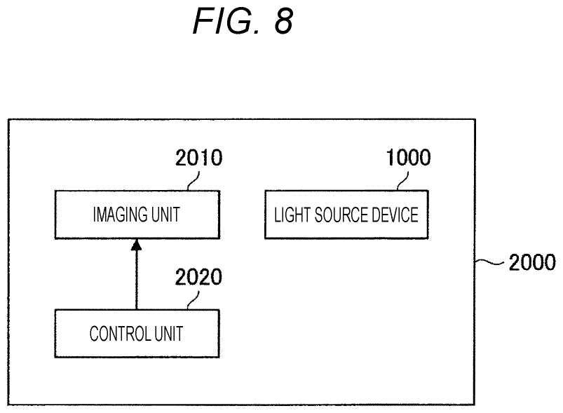

FIG. 8 is a schematic diagram showing the configuration of a surgical observation apparatus 2000 to which the light source device 1000 is applied. The surgical observation apparatus 2000 includes an imaging unit 2010 and a control unit 2020 that controls the imaging unit 2010, in addition to the light source device (light source unit) 1000. In a case where the surgical observation apparatus 2000 is used in an endoscope system, the imaging unit 2010 corresponds to the camera of the endoscope, and the control unit 2020 corresponds to the camera control unit (CCU) that controls the camera of the endoscope.

### 5. Example Configuration of a System for Medical Use

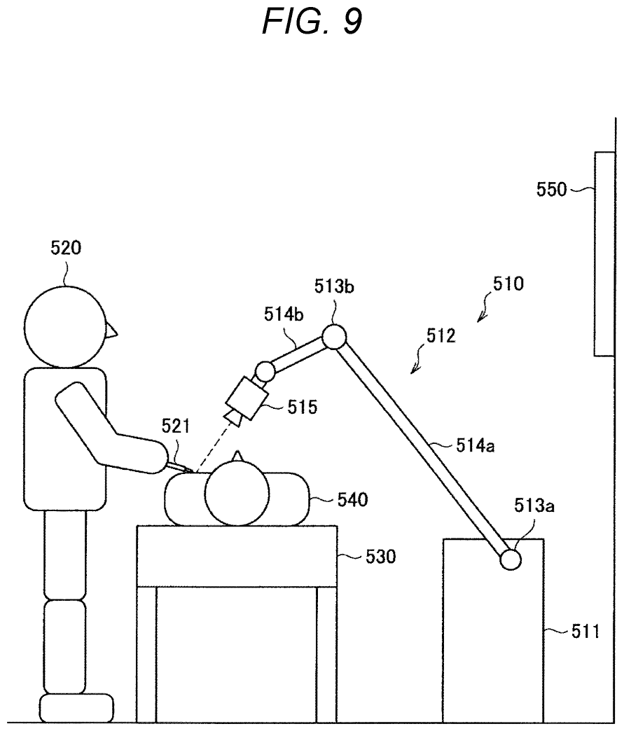

FIG. 9 is an explanatory diagram for explaining a system for medical use to which the light source device 1000 according to an embodiment of the present disclosure is applied. FIG. 9 schematically shows an operation using a robot arm device. Specifically, referring to FIG. 9, a surgeon who is the practitioner (user) 520 is performing surgery on the treatment target (patient) 540 on a surgical table 530, using surgical instruments 521 such as a scalpel, scissors, and forceps, for example. Note that, in the description below, treatment is a general term for various kinds of medical treatment such as surgery and examinations performed by the surgeon who is the user 520 on the patient who is the treatment target 540. Further, in the example shown in FIG. 9, surgery is illustrated as an example of treatment, but the treatment using a robot arm device 510 is not necessarily surgery, but may be other various kinds of treatment, such as examinations and the like using an endoscope, for example.

The robot arm device 510 according to this embodiment is disposed beside the surgical table 530. The robot arm device 510 includes a base unit 511 as the base, and an arm unit 512 extending from the base unit 511. The arm unit 512 includes a plurality of joint portions 513a, 513b, and 513c, a plurality of links 514a and 514b connected by the joint portions 513a and 513b, and an imager unit 515 provided at the end of the arm unit 512. In the example shown in FIG. 9, the arm unit 512 has the three joint portions 513a through 513c, and the two links 514a and 514b, for simplification. In practice, however, the numbers and the shapes of the joint portions 513a through 513c and the links 514a and 514b, the orientation of the drive shafts of the joint portions 513a through 513c, and the like may be set as appropriate so that a desired degree of freedom can be achieved, with the degree of freedom in the positions and the postures of the arm unit 512 and the imager unit 515 being taken into consideration.

The joint portions 513a through 513c have a function of rotatably connecting the links 514a and 514b to each other, and the joint portions 513a through 513c are rotationally driven, so that the driving of the arm unit 512 is controlled. Here, in the description below, the position of each component of the robot arm device 510 means a position (coordinates) in a space defined for drive control, and the posture of each component means an orientation (angle) with respect to an appropriate axis in the space defined for drive control. Further, in the description below, driving of (or drive control on) the arm unit 512 means driving of (or drive control on) the joint portions 513a through 513c, and changing (or controlling changes in) the positions and the postures of the respective components of the arm unit 512 through driving of (or drive control on) the joint portions 513a through 513c.

Various kinds of medical equipment are connected as an end unit to the end of the arm unit 512. In the example shown in FIG. 9, the imager unit 515 is provided as an example of the end unit at the end of the arm unit 512. The imager unit 515 is a unit that acquires an image to be captured (a captured image), and is a camera or the like that can capture a moving image or a still image, for example. As shown in FIG. 9, the postures and the positions of the arm unit 512 and the imager unit 515 are controlled by the robot arm device 510 so that the imager unit 515 provided at the end of the arm unit 512 captures an image of the treatment site of the treatment target 540. Note that the end unit provided at the end of the arm unit 512 is not necessarily the imager unit 515, but may be various types of medical equipment. Examples of the medical equipment include an endoscope, a microscope, a unit having an imaging function such as the imager unit 515 described above, and various kinds of units to be used during treatment, such as various kinds of treatment tools, inspection devices, and the like. In view of this, the robot arm device 510 according to this embodiment can be regarded as a medical robot arm device including medical equipment. Alternatively, a stereo camera including two imager units (camera units) may be provided at the end of the arm unit 512, and imaging may be performed so as to display the imaging target as a three-dimensional image (3D image). Note that the robot arm device 510 including, as the end unit, the imager unit 515 for capturing an image of a treatment site or a camera unit such as the stereo camera is also referred to as a video microscope (VM) robot arm device.

Further, a display device 550 such as a monitor or a display is installed at a position facing the user 520. A captured image of the treatment site imaged by the imager unit 515 is displayed on the display screen of the display device 550. The user 520 performs various kinds of treatment while viewing the captured image of the treatment site displayed on the display screen of the display device 550.

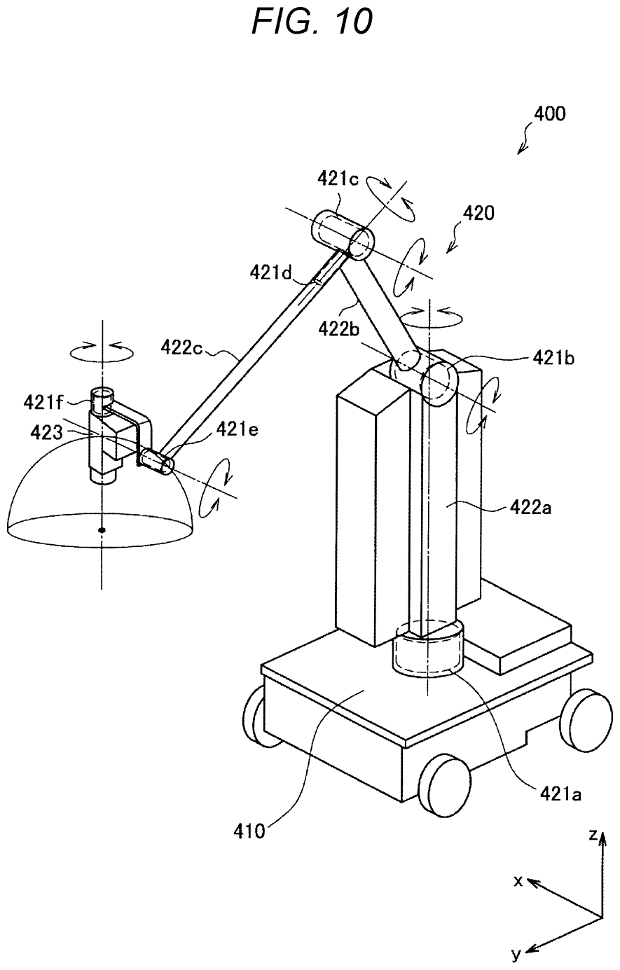

FIG. 10 is a schematic view showing the exterior of the robot arm device shown in FIG. 9. Referring to FIG. 10, a robot arm device 400 according to this embodiment includes a base unit 410 and an arm unit 420. The base unit 410 is the base of the robot arm device 400, and the arm unit 420 extends from the base unit 410. Further, although not shown in FIG. 10, a control unit that comprehensively controls the robot arm device 400 may be provided in the base unit 410, and driving of the arm unit 420 may be controlled by the control unit. The control unit is formed with one of various signal processing circuits such as a central processing unit (CPU) or a digital signal processor (DSP), for example.

The arm unit 420 includes a plurality of joint portions 421a through 421f, a plurality of links 422a through 422c connected to one another by the joint portions 421a through 421f, and an imager unit 423 provided at the end of the arm unit 420.

The links 422a through 422c are rod-like members, one end of the link 422a is connected to the base unit 410 via the joint portion 421a, the other end of the link 422a is connected to one end of the link 422b via the joint portion 421b, and the other end of the link 422b is further connected to one end of the link 422c via the joint portions 421c and 421d. Further, the imager unit 423 is connected to the end of the arm unit 420, which is the other end of the link 422c, via the joint portions 421e and 421f. In this manner, the ends of the plurality of links 422a through 422c are connected to one another by the joint portions 421a through 421f, with the base unit 410 being the fulcrum. Thus, an arm-like shape extending from the base unit 410 is formed.

The imager unit 423 is a unit that acquires an image of the imaging target, and is a camera or the like that captures a moving image or a still image, for example. As the driving of the arm unit 420 is controlled, the position and the posture of the imager unit 423 are controlled. In this embodiment, the imager unit 423 captures an image of a partial region that is a treatment site of the patient's body, for example. However, the end unit provided at the end of the arm unit 420 is not necessarily the imager unit 423, and various kinds of medical equipment may be connected as the end unit to the end of the arm unit 420. In view of this, the robot arm device 400 according to this embodiment can be regarded as a medical robot arm device including medical equipment.

Here, the robot arm device 400 is described below, with the coordinate axes being defined as shown in FIG. 10. Also, the vertical direction, the front-back direction, and the horizontal direction are defined in conjunction with the coordinate axes. That is, the vertical direction with respect to the base unit 410 placed on the floor is defined as the z-axis direction and the vertical direction. Also, the direction that is orthogonal to the z-axis and is the direction in which the arm unit 420 extends from the base unit 410 (which is the direction in which the imager unit 423 is positioned with respect to the base unit 410) is defined as the y-axis direction and the front-back direction. Further, the direction orthogonal to the y-axis and the z-axis is defined as the x-axis direction and the horizontal direction.

The joint portions 421a through 421f rotatably connect the links 422a through 422c to one another. The joint portions 421a through 421f each include an actuator, and have a rotation mechanism that is driven to rotate about a predetermined rotation axis by driving of the actuator. As the rotational driving of each of the joint portions 421a through 421f is controlled, the driving of the arm unit 420, such as extension or retraction (folding) of the arm unit 420, can be controlled, for example. Here, the driving of the joint portions 421a through 421f is controlled by whole body coordinated control and ideal joint control. Further, as described above, the joint portions 421a through 421f according to this embodiment have a rotation mechanism. Therefore, in the description below, drive control on the joint portions 421a through 421f specifically means control on the rotation angle and/or generated torque (the torque to be generated by the joint portions 421a through 421f) of the joint portions 421a through 421f.

The robot arm device 400 according to this embodiment includes six joint portions 421a through 421f, and has six degrees of freedom in driving the arm unit 420. Specifically, as shown in FIG. 10, the joint portions 421a, 421d, and 421f are positioned so that the long-axis direction of each of the connected links 422a through 422c and the imaging direction of the connected imager unit 473 serve as the direction of the rotation axis. The joint portions 421b, 421c, and 421e are positioned so that the x-axis direction that is the direction for changing the connection angle of each of the connected links 422a through 422c and the imager unit 473 in the y-z plane (the plane defined by the y-axis and the z-axis) serves as the direction of the rotation axis. Accordingly, in this embodiment, the joint portions 421a, 421d, and 421f have a function of performing so-called yawing, and the joint portions 421b, 421c, and 421e have a function of performing so-called pitching.

Having such a configuration as the arm unit 420, the robot arm device 400 according to this embodiment has six degrees of freedom in driving the arm unit 420. Thus, the imager unit 423 can be freely moved within the range of movement of the arm unit 420. In FIG. 10, a hemisphere is shown as an example of the range of movement of the imager unit 423. Where the center point of the hemisphere is the imaging center of the treatment site to be imaged by the imager unit 423, the imager unit 423 is moved on the spherical surface of the hemisphere, with the imaging center of the imager unit 423 being fixed at the center point of the hemisphere. Thus, the treatment site can be imaged from various angles.

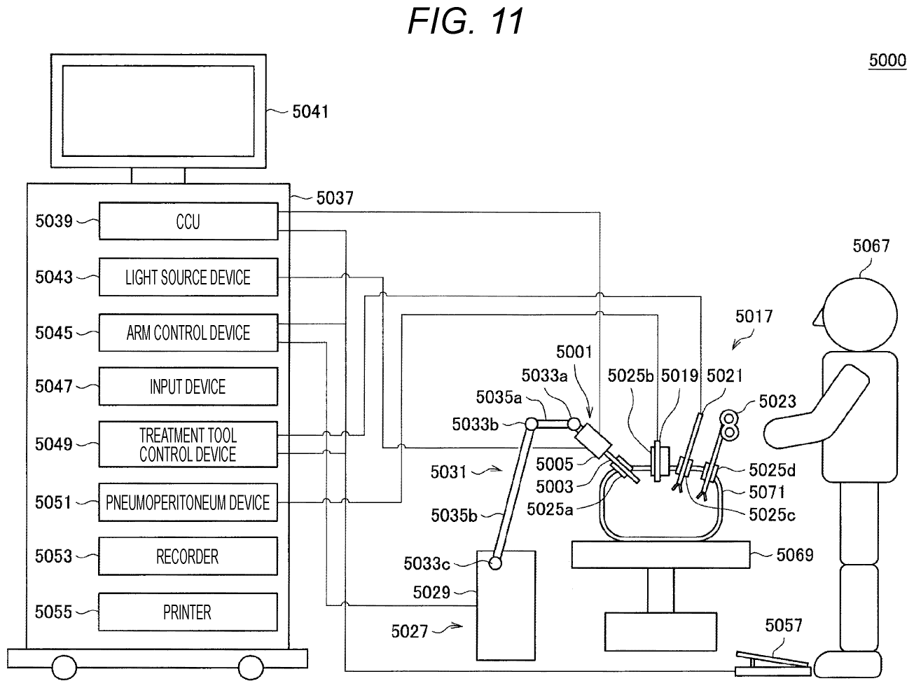

FIG. 11 is a diagram schematically showing an example configuration of an endoscopic surgery system 5000 to which the technology according to the present disclosure can be applied. FIG. 11 shows a situation where an operator (a surgeon) 5067 is performing surgery on a patient 5071 on a patient bed 5069, using the endoscopic surgery system 5000. As shown in the drawing, the endoscopic surgery system 5000 includes an endoscope 5001, other surgical tools 5017, a support arm device 5027 that supports the endoscope 5001, and a cart 5037 in which various kinds of devices for endoscopic surgery are installed.

In endoscopic surgery, the abdominal wall is not cut to open the abdomen, but is punctured with a plurality of cylindrical puncture devices called trocars 5025a through 5025d. Through the trocars 5025a through 5025d, the lens barrel 5003 of the endoscope 5001 and the other surgical tools 5017 are then inserted into a body cavity of the patient 5071. In the example shown in the drawing, a pneumoperitoneum tube 5019, an energy treatment tool 5021, and forceps 5023 are inserted as the other surgical tools 5017 into the body cavity of the patient 5071. Further, the energy treatment tool 5021 is a treatment tool for performing incision and detachment of tissue, blood vessel sealing, or the like, using a high-frequency current or ultrasonic vibration. However, the surgical tools 5017 shown in the drawing are merely an example, and various other surgical tools that are generally used for endoscopic surgery such as tweezers and a retractor, for example, may be used as the surgical tools 5017.

An image of the surgical site in the body cavity of the patient 5071 imaged by the endoscope 5001 is displayed on a display device 5041. The operator 5067 performs treatment such as cutting off the affected site with the energy treatment tool 5021 and the forceps 5023, for example, while viewing the image of the surgical site displayed on the display device 5041 in real time. Note that, although not shown in the drawing, the pneumoperitoneum tube 5019, the energy treatment tool 5021, and the forceps 5023 are supported by the operator 5067 or an assistant or the like during surgery.

(Support Arm Device)

The support arm device 5027 includes an arm unit 5031 extending from a base unit 5029. In the example shown in the drawing, the arm unit 5031 includes joint portions 5033a, 5033b, and 5033c, and links 5035a and 5035b, and is driven under the control of an arm control device 5045. The endoscope 5001 is supported by the arm unit 5031, and its position and posture are controlled.

Thus, the endoscope 5001 can be secured in a stable position.

(Endoscope)

The endoscope 5001 includes a lens barrel 5003 that has a region of a predetermined length from the top end to be inserted into a body cavity of the patient 5071, and a camera head 5005 connected to the base end of the lens barrel 5003. In the example shown in the drawing, the endoscope 5001 is designed as a so-called rigid scope having a rigid lens barrel 5003. However, the endoscope 5001 may be designed as a so-called flexible scope having a flexible lens barrel 5003.

At the top end of the lens barrel 5003, an opening into which an objective lens is inserted is provided. A light source device 5043 is connected to the endoscope 5001, and light generated by the light source device 5043 is guided to the top end of the lens barrel 5003 by a light guide extending inside the lens barrel, and is emitted toward the current observation target in the body cavity of the patient 5071 via the objective lens. Note that the endoscope 5001 may be a forward-viewing endoscope, an oblique-viewing endoscope, or a side-viewing endoscope.

An optical system and an imaging device are provided inside the camera head 5005, and reflected light (observation light) from the current observation target is converged on the imaging device by the optical system. The observation light is photoelectrically converted by the imaging device, and an electrical signal corresponding to the observation light, or an image signal corresponding to the observation image, is generated. The image signal is transmitted as RAW data to a camera control unit (CCU) 5039. Note that the camera head 5005 is made to drive the optical system as appropriate, to achieve a function to adjust magnification and focal length.

Note that, to cope with stereoscopic viewing (3D display) or the like, for example, a plurality of imaging devices may be provided in the camera head 5005. In this case, a plurality of relay optical systems is provided inside the lens barrel 5003, to guide the observation light to each of the imaging devices.

(Various Devices Installed in the Cart)

The CCU 5039 is formed with a central processing unit (CPU), a graphics processing unit (GPU), or the like, and collectively controls operations of the endoscope 5001 and the display device 5041. Specifically, the CCU 5039 performs various kinds of image processing, such as a development process (demosaicing process), for example, for displaying an image based on an image signal received from the camera head 5005. The CCU 5039 supplies the image signal subjected to the image processing, to the display device 5041. The CCU 5039 further transmits a control signal to the camera head 5005, and controls its driving. The control signal may contain information about imaging conditions such as magnification and focal length.

Under the control of the CCU 5039, the display device 5041 displays an image based on the image signal subjected to the image processing by the CCU 5039. In a case where the endoscope 5001 is compatible with high-resolution imaging such as 4K (the number of pixels in a horizontal direction×the number of pixels in a vertical direction: 3840×2160) or 8K (the number of pixels in a horizontal direction×the number of pixels in a vertical direction: 7680×4320), and/or is compatible with 3D display, for example, the display device 5041 may be a display device that is capable of high-resolution display, and/or is capable of 3D display, accordingly. In a case where the endoscope 5001 is compatible with high-resolution imaging such as 4K or 8K, a display device of 55 inches or larger in size is used as the display device 5041, to obtain a more immersive feeling. Further, a plurality of display devices 5041 of various resolutions and sizes may be provided, depending on the purpose of use.

The light source device 5043 is formed with a light source such as a light emitting diode (LED), for example, and supplies the endoscope 5001 with illuminating light for imaging the surgical site.

The arm control device 5045 is formed with a processor such as a CPU, for example, and operates in accordance with a predetermined program, to control the driving of the arm unit 5031 of the support arm device 5027 in accordance with a predetermined control method.

An input device 5047 is an input interface to the endoscopic surgery system 5000. The user can input various kinds of information and instructions to the endoscopic surgery system 5000 via the input device 5047. For example, the user inputs various kinds of information about surgery, such as the patient's physical information and information about the surgical method, via the input device 5047. Further, via the input device 5047, the user inputs an instruction for driving the arm unit 5031, an instruction for changing the imaging conditions (the type of illuminating light, magnification, focal length, and the like) for the endoscope 5001, an instruction for driving the energy treatment tool 5021, and the like, for example.

The input device 5047 is not limited to any particular type, and the input device 5047 may be an input device of any known type. For example, the input device 5047 may be a mouse, a keyboard, a touch panel, a switch, a foot switch 5057, and/or a lever or the like. In a case where a touch panel is used as the input device 5047, the touch panel may be provided on the display surface of the display device 5041.

Alternatively, the input device 5047 is a device worn by a user such as a spectacle-type wearable device or a head-mounted display (HMD), for example, and various inputs are made in accordance with gestures and lines of sight of the user detected by these devices. The input device 5047 also includes a camera capable of detecting motion of the user, and various inputs are made in accordance with gestures and lines of sight of the user detected from a video image captured by the camera. Further, the input device 5047 includes a microphone capable of picking up the voice of the user, and various inputs are made with voice through the microphone. As the input device 5047 is designed to be capable of inputting various kinds of information in a non-contact manner as described above, a user (the operator 5067, for example) in a clean area can operate a device in an unclean area in a non-contact manner. Further, as the user can operate a device without releasing the surgical tool already in his/her hand, user convenience is increased.

A treatment tool control device 5049 controls driving of the energy treatment tool 5021 for tissue cauterization, incision, blood vessel sealing, or the like. A pneumoperitoneum device 5051 injects a gas into a body cavity of the patient 5071 via the pneumoperitoneum tube 5019 to inflate the body cavity, for the purpose of securing the field of view of the endoscope 5001 and the working space of the operator. A recorder 5053 is a device capable of recording various kinds of information about the surgery. A printer 5055 is a device capable of printing various kinds of information relating to the surgery in various formats such as text, images, graphics, and the like.

In the description below, the components particularly characteristic of the endoscopic surgery system 5000 are explained in greater detail.

(Support Arm Device)

The support arm device 5027 includes the base unit 5029 as the base, and the arm unit 5031 extending from the base unit 5029. In the example shown in the drawing, the arm unit 5031 includes the plurality of joint portions 5033a, 5033b, and 5033c, and the plurality of links 5035a and 5035b connected by the joint portion 5033b. For simplicity, FIG. 11 shows the configuration of the arm unit 5031 in a simplified manner. In practice, the shapes, the number, and the arrangement of the joint portions 5033a through 5033c and the links 5035a and 5035b, the directions of the rotation axes of the joint portions 5033a through 5033c, and the like are appropriately set so that the arm unit 5031 can have a desired degree of freedom. For example, the arm unit 5031 is preferably designed to have a degree of freedom equal to or higher than six degrees. This allows the endoscope 5001 to freely move within the movable range of the arm unit 5031. Thus, it becomes possible to insert the lens barrel 5003 of the endoscope 5001 into the body cavity of the patient 5071 from a desired direction.

Actuators are provided for the joint portions 5033a through 5033c, and the joint portions 5033a through 5033c are designed to be able to rotate about a predetermined rotation axis when the actuators are driven. As the driving of the actuators is controlled by the arm control device 5045, the rotation angles of the respective joint portions 5033a through 5033c are controlled, and thus, the driving of the arm unit 5031 is controlled. In this manner, the position and the posture of the endoscope 5001 can be controlled. At this stage, the arm control device 5045 can control the driving of the arm unit 5031 by various known control methods such as force control or position control.

For example, the operator 5067 may make an appropriate operation input via the input device 5047 (including the foot switch 5057), so that the arm control device 5045 appropriately can control the driving of the arm unit 5031 in accordance with the operation input, and the position and the posture of the endoscope 5001 can be controlled. Through this control, the endoscope 5001 at the distal end of the arm unit 5031 can be moved from a position to a desired position, and can be supported in a fixed manner at the desired position after the movement. Note that the arm unit 5031 may be operated by a so-called master-slave mode. In this case, the arm unit 5031 can be remotely operated by the user via the input device 5047 installed at a place away from the operating room.

Alternatively, in a case where force control is adopted, the arm control device 5045 is subjected to external force from the user, and performs so-called power assist control to drive the actuators of the respective joint portions 5033a through 5033c so that the arm unit 5031 moves smoothly with the external force. Because of this, when the user moves the arm unit 5031 while directly touching the arm unit 5031, the arm unit 5031 can be moved with a relatively small force. Thus, it becomes possible to more intuitively move the endoscope 5001 with a simpler operation, and increase user convenience accordingly.

Here, in general endoscopic surgery, the endoscope 5001 is supported by a medical doctor called a scopist. In a case where the support arm device 5027 is used, on the other hand, it is possible to secure the position of the endoscope 5001 with a higher degree of precision without any manual operation. Thus, an image of the surgical site can be obtained in a constant manner, and surgery can be performed smoothly.

Note that the arm control device 5045 is not necessarily installed in the cart 5037. Further, the arm control device 5045 is not necessarily one device. For example, the arm control device 5045 may be provided in each of the joint portions 5033a through 5033c of the arm unit 5031 of the support arm device 5027, and the plurality of arm control devices 5045 may cooperate with one another, to control the driving of the arm unit 5031.

(Light Source Device)

The light source device 5043 supplies the endoscope 5001 with illuminating light for imaging the surgical site. The light source device 5043 is formed with an LED, a laser light source, or a white light source that is a combination of an LED and a laser light source, for example. Here, in a case where the white light source is formed with a combination of RGB laser light sources, the output intensity and the output timing of each color (each wavelength) can be controlled with high precision. Accordingly, the white balance of a captured image can be adjusted at the light source device 5043. Alternatively, in this case, laser light from each of the RGB laser light sources may be emitted onto the current observation target in a time-division manner, and driving of the imaging device of the camera head 5005 may be controlled in synchronization with the timing of the light emission. Thus, images corresponding to the respective RGB colors can be captured in a time-division manner. According to the method, a color image can be obtained without any color filter provided in the imaging device.

Further, the driving of the light source device 5043 may also be controlled so that the intensity of light to be output is changed at predetermined time intervals. The driving of the imaging device of the camera head 5005 is controlled in synchronization with the timing of the change in the intensity of the light, and images are acquired in a time-division manner and are then combined. Thus, a high dynamic range image with no black portions and no white spots can be generated.

Further, the light source device 5043 may also be designed to be capable of supplying light of a predetermined wavelength band compatible with special light observation. In special light observation, light of a narrower band than the illuminating light (or white light) at the time of normal observation is emitted, with the wavelength dependence of light absorption in body tissue being taken advantage of, for example. As a result, so-called narrowband light observation (narrowband imaging) is performed to image predetermined tissue such as a blood vessel in a mucosal surface layer or the like, with high contrast. Alternatively, in the special light observation, fluorescence observation for obtaining an image with fluorescence generated through emission of excitation light may be performed. In fluorescence observation, excitation light is emitted onto body tissue so that the fluorescence from the body tissue can be observed (autofluorescence observation). Alternatively, a reagent such as indocyanine green (ICG) is locally injected into body tissue, and excitation light corresponding to the fluorescence wavelength of the reagent is emitted onto the body tissue so that a fluorescent image can be obtained, for example. The light source device 5043 can be designed to be capable of supplying narrowband light and/or excitation light compatible with such special light observation.

(Camera Head And CCU)

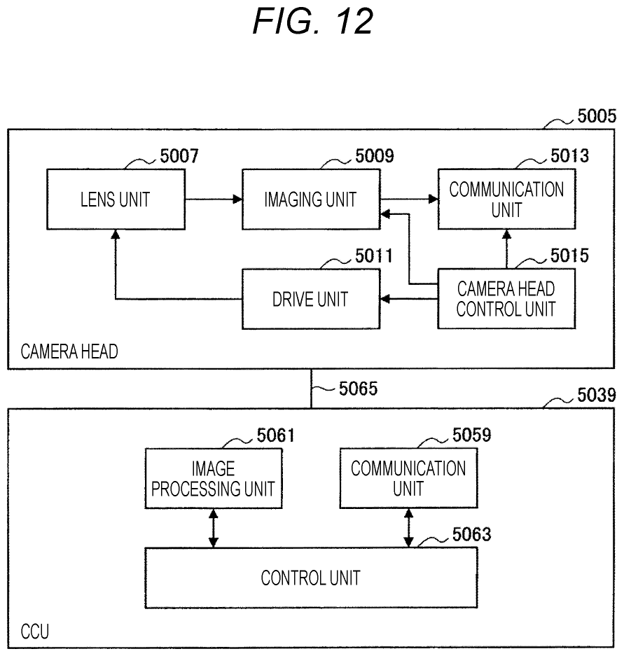

Referring now to FIG. 12, the functions of the camera head 5005 and the CCU 5039 of the endoscope 5001 are described in greater detail. FIG. 12 is a block diagram showing an example of the functional configurations of the camera head 5005 and the CCU 5039 shown in FIG. 11.

As shown in FIG. 12, the camera head 5005 includes, as its functions, a lens unit 5007, an imaging unit 5009, a drive unit 5011, a communication unit 5013, and a camera head control unit 5015. Meanwhile, the CCU 5039 includes, as its functions, a communication unit 5059, an image processing unit 5061, and a control unit 5063. The camera head 5005 and the CCU 5039 are connected by a transmission cable 5065 so that bidirectional communication can be performed.

First, the functional configuration of the camera head 5005 is described. The lens unit 5007 is an optical system provided at the connecting portion with the lens barrel 5003. Observation light captured from the top end of the lens barrel 5003 is guided to the camera head 5005, and enters the lens unit 5007. The lens unit 5007 is formed with a combination of a plurality of lenses including a zoom lens and a focus lens. The optical characteristics of the lens unit 5007 are adjusted so as to collect the observation light onto the light receiving surface of the imaging device of the imaging unit 5009. Further, the zoom lens and the focus lens are designed so that the positions thereof on the optical axis can move to adjust the magnification and the focal point of a captured image.

The imaging unit 5009 is formed with an imaging device, and is disposed at a stage after the lens unit 5007. The observation light having passed through the lens unit 5007 is gathered on the light receiving surface of the imaging device, and an image signal corresponding to the observation image is generated through photoelectric conversion. The image signal generated by the imaging unit 5009 is supplied to the communication unit 5013.

The imaging device forming the imaging unit 5009 is an image sensor of a complementary metal oxide semiconductor (CMOS) type, for example, and the image sensor to be used here has a Bayer array and is capable of color imaging. Note that the imaging device may be an imaging device compatible with capturing images of high resolution such as 4K or higher, for example. As a high-resolution image of the surgical site is obtained, the operator 5067 can grasp the state of the surgical site in greater detail, and proceed with the surgery more smoothly.

Further, the imaging device of the imaging unit 5009 is designed to include a pair of imaging devices for acquiring right-eye and left-eye image signals compatible with 3D display. As the 3D display is conducted, the operator 5067 can grasp more accurately the depth of the body tissue at the surgical site. Note that, in a case where the imaging unit 5009 is of a multiple-plate type, a plurality of lens units 5007 is provided for the respective imaging devices.

Further, the imaging unit 5009 is not necessarily provided in the camera head 5005. For example, the imaging unit 5009 may be provided immediately behind the objective lens in the lens barrel 5003.

The drive unit 5011 is formed with an actuator, and, under the control of the camera head control unit 5015, moves the zoom lens and the focus lens of the lens unit 5007 by a predetermined distance along the optical axis. With this arrangement, the magnification and the focal point of the image captured by the imaging unit 5009 can be adjusted as appropriate.

The communication unit 5013 is formed with a communication device for transmitting and receiving various kinds of information to and from the CCU 5039. The communication unit 5013 transmits the image signal obtained as RAW data from the imaging unit 5009 to the CCU 5039 via the transmission cable 5065. At this stage, to display a captured image of the surgical site with low latency, the image signal is preferably transmitted through optical communication. The operator 5067 performs surgery while observing the state of the affected site through the captured image during the operation. Therefore, for the operator 5067 to perform safe and reliable surgery, a moving image of the surgical site should be displayed in as real time as possible. In a case where optical communication is performed, a photoelectric conversion module that converts an electrical signal into an optical signal is provided in the communication unit 5013. The image signal is converted into an optical signal by the photoelectric conversion module, and is then transmitted to the CCU 5039 via the transmission cable 5065.

The communication unit 5013 also receives, from the CCU 5039, a control signal for controlling driving of the camera head 5005. The control signal includes information about imaging conditions, such as information for specifying the frame rate of captured images, information for specifying the exposure value at the time of imaging, and/or information for specifying the magnification and the focal point of captured images, for example. The communication unit 5013 supplies the received control signal to the camera head control unit 5015. Note that the control signal from the CCU 5039 may also be transmitted through optical communication. In this case, a photoelectric conversion module that converts an optical signal into an electrical signal is provided in the communication unit 5013, and the control signal is converted into an electrical signal by the photoelectric conversion module, and is then supplied to the camera head control unit 5015.

Note that the above imaging conditions such as the frame rate, the exposure value, the magnification, and the focal point are automatically set by the control unit 5063 of the CCU 5039 on the basis of the acquired image signal. That is, the endoscope 5001 has a so-called auto-exposure (AE) function, an auto-focus (AF) function, and an auto-white-balance (AWB) function.

The camera head control unit 5015 controls the driving of the camera head 5005, on the basis of a control signal received from the CCU 5039 via the communication unit 5013. For example, the camera head control unit 5015 controls the driving of the imaging device of the imaging unit 5009 on the basis of the information for specifying the frame rate of captured images and/or the information for specifying the exposure at the time of imaging. Alternatively, the camera head control unit 5015 appropriately moves the zoom lens and the focus lens of the lens unit 5007 via the drive unit 5011, on the basis of the information for specifying the magnification and the focal point of captured image, for example. The camera head control unit 5015 may further have a function to store information for identifying the lens barrel 5003 and the camera head 5005.

Note that components such as the lens unit 5007 and the imaging unit 5009 are disposed in a hermetically sealed structure with high airtightness and waterproofness, so that the camera head 5005 can be tolerant of autoclave sterilization.

Next, the functional configuration of the CCU 5039 is described. The communication unit 5059 is formed with a communication device for transmitting and receiving various kinds of information to and from the camera head 5005. The communication unit 5059 receives an image signal transmitted from the camera head 5005 via the transmission cable 5065. At this stage, the image signal can be transmitted preferably through optical communication, as described above. In this case, to cope with optical communication, the communication unit 5059 includes a photoelectric conversion module that converts an optical signal into an electrical signal. The communication unit 5059 supplies the image signal converted into the electrical signal to the image processing unit 5061.

Further, the communication unit 5059 also transmits a control signal for controlling the driving of the camera head 5005, to the camera head 5005. The control signal may also be transmitted through optical communication.

The image processing unit 5061 performs various kinds of image processing on an image signal that is RAW data transmitted from the camera head 5005. Examples of the image processing include various kinds of known signal processing, such as a development process, an image quality enhancement process (a band emphasizing process, a super-resolution process, a noise reduction (NR) process, a camera shake correction process, and/or the like), and/or an enlargement process (an electronic zooming process), for example. The image processing unit 5061 further performs a detection process on the image signal, to perform AE, AF, and AWB.

The image processing unit 5061 is formed with a processor such as a CPU or a GPU. As this processor operates in accordance with a predetermined program, the above described image processing and the detection process can be performed. Note that, in a case where the image processing unit 5061 is formed with a plurality of GPUs, the image processing unit 5061 appropriately divides information about an image signal, and the plurality of GPUs perform image processing in parallel.

The control unit 5063 performs various kinds of control relating to imaging of the surgical site with the endoscope 5001 and display of the captured image. For example, the control unit 5063 generates a control signal for controlling the driving of the camera head 5005. In a case where the imaging conditions have already been input by the user at this stage, the control unit 5063 generates the control signal on the basis of the input made by the user. Alternatively, in a case where the endoscope 5001 has an AE function, an AF function, and an AWB function, the control unit 5063 generates a control signal by appropriately calculating an optimum exposure value, an optimum focal length, and an optimum white balance in accordance with a result of the detection process performed by the image processing unit 5061.

The control unit 5063 also causes the display device 5041 to display an image of the surgical site, on the basis of the image signal subjected to the image processing by the image processing unit 5061. In doing so, the control unit 5063 may recognize the respective objects shown in the image of the surgical site, using various image recognition techniques. For example, the control unit 5063 can detect the shape, the color, and the like of the edges of an object shown in the image of the surgical site, to recognize the surgical tool such as forceps, a specific body site, bleeding, the mist at the time of use of the energy treatment tool 5021, and the like. When causing the display device 5041 to display the image of the surgical site, the control unit 5063 may cause the display device 5041 to superimpose various kinds of surgery aid information on the image of the surgical site on the display, using a result of the recognition. As the surgery aid information is superimposed and displayed, and thus, is presented to the operator 5067, the operator 5067 can proceed with safer surgery in a more reliable manner.

The transmission cable 5065 connecting the camera head 5005 and the CCU 5039 is an electrical signal cable compatible with electric signal communication, an optical fiber compatible with optical communication, or a composite cable thereof.

Here, in the example shown in the drawing, communication is performed in a wired manner using the transmission cable 5065. However, communication between the camera head 5005 and the CCU 5039 may be performed in a wireless manner. In a case where communication between the two is performed in a wireless manner, there is no need to install the transmission cable 5065 in the operating room. Thus, it is possible to avoid a situation in which movement of the medical staff in the operating room is hindered by the transmission cable 5065.

An example of the endoscopic surgery system 5000 to which the technique according to the present disclosure can be applied has been described above. Note that, although the endoscopic surgery system 5000 has been described as an example herein, systems to which the technology according to the present disclosure can be applied are not limited to such an example. For example, the technology according to the present disclosure may be applied to a flexible endoscope system for inspection and a microscopic surgery system.

The technology according to the present disclosure can be suitably applied to systems for medical use as shown in FIGS. 9 through 12. Specifically, in the system shown in FIGS. 9 and 10, the light source device 1000 is installed in the base unit 511 (or the base unit 410), for example. The light guide 195 of the light source device 1000 is guided to the imager unit 515 (or the imager unit 423) through the inside or the outside of the plurality of links 514a and 514b (or the plurality of links 422a through 422c). The imager unit 515 (or the imager unit 423) corresponds to the imaging unit 2010 shown in FIG. 8.

The light source device 1000 according to the present disclosure can also be suitably applied to the light source device 5043 of the system shown in FIGS. 11 and 12. Light generated by the light source device 5043 is guided to the end of the lens barrel 5003 by the light guide 195. The imaging unit 5009 corresponds the imaging unit 2010 shown in FIG. 8.

### 6. Variation of Control

In the system shown in FIGS. 11 and 12, the light source device 5043 can be controlled with information input from the input device 5047. The control on the light source device 5043 may be performed via the CCU 5039. Accordingly, it is possible to perform various operations, such as special light zooming, mode switching, and fluorescent agent selection, by operating the input device 5047, instead of operating various switches provided on the exterior of the light source device 1000 as shown in FIG. 7. Note that the input device 5047 may be a portable terminal such as a tablet device, or may be a device that wirelessly communicates with the CCU 5039 or the light source device 5043.

Further, various operations such as special light zooming can be performed with the foot switch 5057. Thus, various operations can be performed while treatment is being conducted, and convenience during treatment can be further enhanced.

Also, image processing may be performed on an image captured by the imaging unit 5009 of the endoscope 5001, so that the shape of an organ (such as the stomach or the liver, for example) or a tumor is recognized, and zooming is performed to emit the special light only onto the portion of the organ. In recognizing the shape of an organ or a tumor, machine learning by artificial intelligence (AI) can be used.

Further, if the special light is continuously emitted, the observation target will be damaged. Therefore, in a case where damage is detected as a result of image processing performed on an image captured by the imaging unit 5009 of the endoscope 5001, panning may be automatically performed, and the zooming may be switched to the wide side, to reduce the damage. At this stage, the damage can also be determined by time integration. For example, in a case where the degree of damage after a certain period of time is higher than the previous degree of damage after the same certain period of time, it is determined that damage has been caused by the special light, and panning is automatically performed.

The irradiation region of the special light can be changed in a stepwise manner or in a continuous manner. In a case where the irradiation region is changed in a stepwise manner, the irradiation region is instantaneously changed to a preset predetermined magnification in one operation, for example. In a case where the irradiation region is changed in a continuous manner, on the other hand, the irradiation region is continuously reduced or enlarged by long-pressing of an operating member or the like. These methods for changing the irradiation region can be switched as appropriate, depending on the environment in which the light source device 1000 is used, the user's preference, and the like.

As described above, according to this embodiment, only the irradiation region of the special light can be enlarged or reduced when the special light and the visible light are emitted onto the observation target. Accordingly, the special light is emitted only onto the portion the user wishes to closely observe, so that the portion to be closely observed can be recognized without fail. Also, as the special light is not emitted onto the portions other than the portion the user wishes to closely observe, it is possible to reduce damage to the observation target.

Further, it is possible to strengthen the excitation light in the region to be observed with the special light at the central portion while observing the entire region with visible light. As a result, the fluorescence generated by the excitation light can be strengthened. Thus, it becomes easier to check a fluorescent image of the central portion while checking the surrounding condition.

Furthermore, as the irradiation region of the excitation light can be changed, the special light is initially emitted onto the entire region, so that the site to be closely observed can be easily searched for. When the target is spotted, the irradiation region of the excitation light can be set to the central portion, aiming at the target. Accordingly, it is possible to increase the intensity of fluorescence at the site on which surgery is to be performed without any change in the output of illumination light, and thus, surgery can be easily performed.

While preferred embodiments of the present disclosure have been described above with reference to the accompanying drawings, the technical scope of the present disclosure is not limited to those examples. It is apparent that those who have ordinary skills in the technical field of the present disclosure can make various changes or modifications within the scope of the technical spirit claimed herein, and it should be understood that those changes or modifications are within the technical scope of the present disclosure.

Furthermore, the effects disclosed in this specification are merely illustrative or exemplary, but are not restrictive. That is, the technology according to the present disclosure may achieve other effects obvious to those skilled in the art from the description in the present specification, in addition to or instead of the effects described above.

Note that the configurations described below are also within the technical scope of the present disclosure.

(1)

A surgical observation apparatus including:

a light source unit that includes: a first light source that emits observation light for observing an operative field; a second light source that emits special light in a wavelength band different from the first light source; and an optical system capable of changing an emission angle of the special light with respect to the operative field, the light source unit emitting the observation light and the special light onto the operative field from the same emission port; and

an imaging unit that captures an image of the operative field illuminated by the light source unit.

(2)

The surgical observation apparatus according to (1), in which

the light source unit includes a plurality of light sources in wavelength bands different from one another, and

the second light source is selected from the plurality of light sources.

(3)

The surgical observation apparatus according to (2), in which the first light source includes at least two light sources of the plurality of light sources not selected as the second light source.

(4)

The surgical observation apparatus according to (2) or (3), in which the optical system is capable of changing the emission angle with respect to the operative field, at least for each light source that can be selected as the second light source from the plurality of light sources.

(5)

The surgical observation apparatus according to any one of (1) to (5), in which the first light source and the second light source each include

at least one of a red laser light source that generates red light, a green laser light source that generates green light, a blue laser light source that generates blue light, a violet laser light source that generates violet light, and an infrared laser light source that generates infrared light.

(6)

The surgical observation apparatus according to (5), in which the first light source combines at least the red light, the green light, and the blue light, to emit the observation light.

(7)

The surgical observation apparatus according to (5) or (6), in which the second light source emits the red light, the green light, the blue light, the violet light, or the infrared light, as the special light.

(8)

The surgical observation apparatus according to (7), in which the second light source emits the violet light or the infrared light as the special light.

(9)

The surgical observation apparatus according to any one of (1) to (8), in which the optical system includes a lens that refracts the special light, and changes the emission angle by moving the lens in an optical axis direction.

(10)

The surgical observation apparatus according to any one of (1) to (8), in which the optical system includes a mirror that reflects the special light, and changes the emission angle by changing a region of the mirror.

(11)

The surgical observation apparatus according to any one of (1) to (10), in which the emission angle is made smaller by the optical system, and the special light is emitted onto a region narrower than the observation light.

(12)

The surgical observation apparatus according to any one of (1) to (11), in which the emission angle is made smaller by the optical system, and the special light is emitted onto a central portion of the operative field.

(13)

The surgical observation apparatus according to any one of (1) to (12), further including an input unit that receives an input of control information for changing the emission angle by controlling the optical system.

(14)

A surgical observation method including:

emitting observation light for observing an operative field;

emitting special light in a wavelength band different from the observation light;

emitting the observation light and the special light onto the operative field from the same emission port;

changing an emission angle of the special light with respect to the operative field; and

capturing an image of the operative field illuminated by the observation light and the special light.

(15)

A surgical light source device including:

a first light source that emits observation light for observing an operative field;

a second light source that emits special light in a wavelength band different from the first light source; and

an optical system capable of changing an emission angle of the special light with respect to the operative field,

in which the observation light and the special light are emitted onto the operative field from the same emission port.

(16)

A surgical light irradiation method including:

emitting observation light for observing an operative field;

emitting special light in a wavelength band different from the observation light;

emitting the observation light and the special light onto the operative field from the same emission port; and

changing an emission angle of the special light with respect to the operative field.

### REFERENCE SIGNS LIST

100 Red light source
110 Yellow light source
120 Green light source
130 Blue light source
140 Violet light source
150 Infrared light source
190 Zoom optical system
200, 202, 204, 206, 208, 209 Zoom split mirror
310 Operation unit
320 Communication connector
1000 Light source device
2000 Surgical observation apparatus
2010 Imaging unit

1. A surgical observation apparatus comprising:
a light source unit that includes: a first light source that emits observation light for observing an operative field; a second light source that emits special light in a wavelength band different from the first light source; and an optical system capable of changing an emission angle of the special light with respect to the operative field, the light source unit emitting the observation light and the special light onto the operative field from the same emission port; and
an imaging unit that captures an image of the operative field illuminated by the light source unit.

2. The surgical observation apparatus according to claim 1, wherein
the light source unit includes a plurality of light sources in wavelength bands different from one another, and
the second light source is selected from the plurality of light sources.

3. The surgical observation apparatus according to claim 2, wherein the first light source includes at least two light sources of the plurality of light sources not selected as the second light source.

4. The surgical observation apparatus according to claim 2, wherein the optical system is capable of changing the emission angle with respect to the operative field, at least for each light source that can be selected as the second light source from the plurality of light sources.

5. The surgical observation apparatus according to claim 1, wherein the first light source and the second light source each include
at least one of a red laser light source that generates red light, a green laser light source that generates green light, a blue laser light source that generates blue light, a violet laser light source that generates violet light, and an infrared laser light source that generates infrared light.

6. The surgical observation apparatus according to claim 5, wherein the first light source combines at least the red light, the green light, and the blue light, to emit the observation light.

7. The surgical observation apparatus according to claim 5, wherein the second light source emits the red light, the green light, the blue light, the violet light, or the infrared light, as the special light.

8. The surgical observation apparatus according to claim 7, wherein the second light source emits the violet light or the infrared light as the special light.

9. The surgical observation apparatus according to claim 1, wherein the optical system includes a lens that refracts the special light, and changes the emission angle by moving the lens in an optical axis direction.

10. The surgical observation apparatus according to claim 1, wherein the optical system includes a mirror that reflects the special light, and changes the emission angle by changing a region of the mirror.

11. The surgical observation apparatus according to claim 1, wherein the emission angle is made smaller by the optical system, and the special light is emitted onto a region narrower than the observation light.

12. The surgical observation apparatus according to claim 1, wherein the emission angle is made smaller by the optical system, and the special light is emitted onto a central portion of the operative field.

13. The surgical observation apparatus according to claim 1, further comprising an input unit that receives an input of control information for changing the emission angle by controlling the optical system.

14. A surgical observation method comprising:
emitting observation light for observing an operative field;
emitting special light in a wavelength band different from the observation light;
emitting the observation light and the special light onto the operative field from the same emission port;
changing an emission angle of the special light with respect to the operative field; and
capturing an image of the operative field illuminated by the observation light and the special light.

15. A surgical light source device comprising:
a first light source that emits observation light for observing an operative field;
a second light source that emits special light in a wavelength band different from the first light source; and
an optical system capable of changing an emission angle of the special light with respect to the operative field,
wherein the observation light and the special light are emitted onto the operative field from the same emission port.

16. A surgical light irradiation method comprising:
emitting observation light for observing an operative field;
emitting special light in a wavelength band different from the observation light;
emitting the observation light and the special light onto the operative field from the same emission port; and
changing an emission angle of the special light with respect to the operative field.
